SEQUENCE LISTING (16 sequences)

SEQ ID NO: 1 - protein, 107 aa, Artificial Sequence [sequence omitted]

SEQ ID NO: 2 - DNA, 287 bp, Homo sapiens
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctcc

SEQ ID NO: 3 - protein, 95 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 4 - DNA, 36 bp, Homo sapiens
ctcactttcggcggagggaccaaggtggagatcaaa

SEQ ID NO: 5 - protein, 12 aa, Homo sapiens
LTFGGGTKVEIK

SEQ ID NO: 6 - DNA, 4731 bp, Oryctolagus cuniculus
cagagtgaggggccctcagtcagagcccagaagcgaacctccctgcaggggagtgcggctccaccagggggcgcgcaagacacactgagtccagattctggtcctgctcaggcacagagggagatgaagccctggtttcctgtcagggatttgggtttcctctcctacagttttgagggaagacttctacttcaggattctgtcccttacactgacgccactgacgtagagaagctttatgtgacaggaagaagtcttaaatggacacattcaggtttgtgaacgatgacagtggtcaccagggtcagagatgtgagacaatcaggggacctcgtgagtgttgtccagtctgactcaggacagcgttccaggacactgccgactataagacaattttagttttccttcaaacccagacagacccgagccagagtcttgatttgctacctcagttttataagtcgtcccatcctcctgtcagagcagcctgtgtgctgcactgaaccaaccgtcttattttaactgtgggtgttctgcattcactttctacagactttatacccaatctccatttccctttaatcctgaaacagtctgtggtccctgtgtgcacttccagctgatctggttctgctccagtgtcgtgggaacccctgcacctgctgtggttcagggagagcagaaagtgggacacagagcggctgtgcactctggggctggagtccacatcacggggagctgtgtctgtggctccagcatgatgcccagtgtcctgaggctgaatcacagctgcaggaagagccagtcccagagaccatgtgtccagagtccctgctcttcactccatgtctatgctgactggggtcatcaatccatttgtgttctttcatcagtattagagagggagacacccaggctgcatatcacattgctggatggagcccatttgcacgctttcccatacttgtactgacagtgcaccctggaagacagtacatgatggatctgtatgaggccccatcaccaaggtgggaacactgatgcagtttcatgctcctgggttccagctgacaagcttgagtccactgcctttatttatcatctccataaaacatggccaacacaacaccagtattcaaagatcaatcgtcatgattggccatcgttgctatttcatattcctaaatgtttgataaataatattgagtatagctctatgaggtacaatgatatgtgtggacaaaatgaatgcatattgagtggaattatgaagtcaggcttgatgacacattcataccacaacacatgtttttggtggagttcacctgtctcctgctcttaagagatttccactcacaatattctcttaacctctgtcaccatgctgtgcagtagatccctcagtgtgtggagcctgtgggactaaaactctgaccttgctccagcgtctcctctgttcctccctcgctcagactttggtcttcaccattgtacttcctgcctctgtgccttcagccttttatcccatgtaggtagcatcacatattatgtgtcttcctggcctggatgcttagcagagtgtacttttactgacatattatcttaggcacattattctccgggatcatccaagttgtccaaaatcacagaatttcttactctgtactgcttttaaacttccttttaaaatgtaactttgtccatttctttcattttccattcttgcttccatgagatgtcactggatcatatgtgtgaactcttttgaatggcggtgctttaaatgaacaaggaactgcagaggtgtttgccacagtgactgcagatcccgtggacacctccctgggatttcatattcattgtaccatgcaatggtgcttgaagatcatgtaataattctgtttctagtttttgatgcttttccataatggttttactaattaacctgacacagacttgtaccagggttatcatctctctctacctccataaaacatgtcagctctggtatgtctgataatagtcattctaacaggagggatgggatgtctcattgtgcttctaacatggacatacatgatggtcctagatttgtagcattttatatgtttgtctttatatcttcttttgaaatttatagatgtaacataaattttaaaatttatcattttattggccagcgcatggctcactaggctaatcctccacctgcggtgactgcaacccagtttctagtctcggtcgggcgccggattctgtcccggttgcccctcttctaggccagctctctactgtggcccgggagtgcagtggaggatggcccaagagcttgtgccctgcaccccatgggagaccaggagaagcacctgactcctggcttcggatcagcgtggtgcaccggttacagcggccattggagggtgaaccaacggtaaaggaagacatttctctctgtctctctctctgtcactgtccactctgcttgtcaaaaaagtgtaccattttatttaaaaatttgtctttatatatttgtccaaaaccacagagagactgagactgacagtgatcttctaattcctggtcacttctcagatggctaccaaagattcagctgggtcaggcgacagccaggtgcttggaattctgtccagggctcccacgtgggactacttggaacttcacctatggcctcccagaggggcttaggaaggaatatgagactggaaccaaactaggacacagcactcagcatcctcatacaggagcaggaatccatcaggctcactccagtgctgctcctgtcaccaccttatcatcactgcatttaaggaattgatttcttcccttgggattcagttgtttgagatcttcaacattaggaaataggctttcaatcatatgtataattaatcttcaatttattttaagtatctattttattttagagacagtgttatagcaagagatgaagagaccaacagatggagagagaagcttctgtccagtggttcacttcgtgaatggctgcaaaggctatgatggctgagcctaaagccaggagcctggaacttcctccagtttcccacagggctggaaggggacaaacactttggtcatggtcgacagtgatcaccattgcaatgcctacatctgtgagttcaattgtggactgcatatacaaataatgtcacatactatgtgtccacatctacctggatcatttcttttagtatgtatgctccaggctgacccatgtttcctactgacagaacgacctccttcttaatactgaataatattccatgtccaaatgtgaaagaatccattcactgttctgtgaacacttgcttaattgtagactgaactattatgaacacagctgcattacctgaacccaggactgcgggtgaattgtgacacagtgacttctacccagtgcttgagctgaactcagagtcatgattacagaacagggaagagggggaacacagggcagctgataacaggacgcaaagcaccactcacaggaaggaataattctgcctacaccacagtgggctgactcagttcaccacaacccatgacaggttccacaaataactagaactgaggtctccaggcctcccaacagagtgatggaaaggaggtggaaatgtgcatgacactgagttcatctgtgcccatttctaccatgcacattccaatagcacacaaaccccactaccatgtacagtgttctgtgttcaggaacagttttaaactaaagcaatggatgaataaatgaacaacacatagttaaccttaaaaaatcaacttgcagtccttcatctatttaaaagtgggcagcaggcattaggacaaaattggctaagtccccactagggtcacccgtgttcccatcagagaactgactgagcccagcctcacggcttcccacacttttccctcataatgcatcatgggaaactgtagatgacactacaggcaaaggagcccctaccaccgacgttggaattacagacacagtttcttgcttcagggttcagaaagactaactcctaggggtcataggcatttgggatgtctagggctctcaacccaaaggcagaaatctcccattgtcaccttttctgtccatctctacatttctgtaactctcaccccttctatctctatctctgtcccaatgtctttcatatagatgataaggaactaatgggatttaaataaatgaggaacatgatattgctaacactggatattaagggtgcacacatattaaaatgaaacaaggtatctgccttcaactttttaaaataattataacataaaaattcatatgatctgaatcatatcacagccatcaccgtacacccccaggtcaccacatctgccctgggcgctgtcctgtctgaggcgtctgaccccatgcctgctatataggggcagctcatgcaaatggggcctccctgtgcccatgaaaaccagcccagccctcaccctgcagctctggcacaggagctccagccccaggactcccaggtgtccactcagtgatcgcactcaacacagacgctcaccatggagactgggctgcgctggcttctcctggtcgctgtgctcaaaggtaatgatggggaacgcgggacactgagtctgggagaggatgtgagtgagagacacagagagtgtgagtgacagtgtcctgaccatgtcgtctgtgtttgcagg

SEQ ID NO: 7 - DNA, 37 bp, Artificial Sequence
  primer
aagcttgccaccatggagactgggctgcgctggcttc

SEQ ID NO: 8 - DNA, 21 bp, Artificial Sequence
  primer
ccattggtgagggtgcccgag

SEQ ID NO: 9 - DNA, 20 bp, Artificial Sequence
  primer
atggacatgagggtccccgc

SEQ ID NO: 10 - DNA, 24 bp, Artificial Sequence
  primer
gatttcaactgctcatcagatggc

SEQ ID NO: 11 - protein, 368 aa, Homo sapiens
MPGGCSRGPAAGDGRLRLARLALVLLGWVSSSSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTSAAALEVLFQGPGT

SEQ ID NO: 12 - protein, 107 aa, Artificial Sequence
  anti-TPBG antibody 051 VL
DIQMTQSPSSVSASVGDRVTITCRASQGIYSWLAWYQQKPGKAPKLLIYAASSLQSGVPSRFSGSGSGTDFTLTISSLQPEDFATYYCQQSDSPPYTFGQGTKLEIK

SEQ ID NO: 13 - protein, 107 aa, Artificial Sequence
  anti-TPBG antibody 091 VL
DIQMTQSPSSLSASVGDRVTITCQASQDISNYLNWYQQKPGKAPKLLIYAASTLQIGVPSRFSGSGSGTDFTFTISSLQPEDFATYYCQQANSFPLTFGGGTKVEIK

SEQ ID NO: 14 - protein, 107 aa, Artificial Sequence
  anti-TPBG antibody 097 VL
DIQMTQSPSSLSASVGDRVTITCRASQSISSYLNWYQQKPGKAPKLLIYAASSLQSGVPSRFSGSGSGTDFTLTISSLQPEDFATYYCQQSDSFPLTFGGGTKVEIK

SEQ ID NO: 15 - protein, 18 aa, Homo sapiens
MDMRVPAQLLGLLLLWLR

SEQ ID NO: 16 - DNA, 124 bp, Gallus gallus
gtactcgttgcgcccggtcggggactgtgggcacggggctctgtcccattgctgcgcgggcagggctgtgcgtgcggggccgtcactgattgccgttttctcccctctctcctctccctctcca

(19) United States
(12) Patent Application Publication
(10) Pub. No.: US 2019/0100771 A1
(43) Pub. Date: Apr. 4, 2019

(54) Transgenic rabbit with common light chain

(71) Applicant: Hoffmann-La Roche Inc., Little Falls, NJ (US)

(72) Inventor: Josef Platzer, Geretsried (DE)

(73) Assignee: Hoffmann-La Roche Inc., Little Falls, NJ (US)

(21) Appl. No.: 15/964,791

(22) Filed: Apr. 27, 2018

Related U.S. Application Data
(63) Continuation of application No. PCT/EP2016/075882, filed on Oct. 27, 2016.

(30) Foreign Application Priority Data
Mar. 29, 2016 (EP) 16162580.1

(51) Int. Cl. C12N 15/85; C07K 16/18; A01K 67/027

(52) U.S. Cl. CPC C12N 15/8509; C07K 16/18; A01K 67/0275; C12N 2015/8518; A01K 2267/01; C07K 2317/515; C07K 2317/24; A01K 2217/206; A01K 2227/107; C07K 2317/14

(57) ABSTRACT

Herein is reported a transgenic vector comprising a humanized light chain locus, wherein said humanized light chain locus comprises (a) a V gene segment derived from human light chain V segment IGKV1-39-01, (b) 3′ proximal to said light chain gene segment a promoter, and (c) 5′ proximal to said light chain gene segment at least a fragment of the human IGKJ4 J-element.

Herein is reported a common light chain locus useful for the generation of transgenic rabbits producing human antibodies. Also reported herein is a common light chain variable domain amino acid sequence, multispecific antibodies comprising the common light chain variable domain and transgenic rabbits comprising the respective common light chain locus.

### BACKGROUND OF THE INVENTION

The production of multispecific antibodies is hampered by the problem of chain mispairing resulting in un-paired and mispaired by-product formation. Depending on the chosen format a not neglectable number and amount of these by-products can be formed.

Different approaches for addressing this problem have been developed.

To reduce heavy chain mispairing the knobs-into-hole technology (see e.g. Ridgway, J. B., et al. Prot. Eng. 9 (1996) 617-621) or the CrossMab format (see e.g. Schaefer, W., et al. Proc. Natl. Acad. Sci USA 108 (2011) 11187-11192) have been reported.

To reduce light chain mispairing a common light chain can be employed. This approach inherently requires that for both binding sites each formed by a pair of an antibody heavy chain variable domain and an antibody light chain variable domain the same antibody light chain variable domain has to be used.

Non-human animals comprising a human immunoglobulin locus can be used to produce monospecific antibodies having a common light chain. The human immunoglobulin locus in such animals generally comprises a reduced and limited number of heavy chain germline genes, rearranged germline heavy chain genes or heavy chain V gene segments and a single light chain gene. When such a non-human animal is immunized in order to produce antibodies the elicited immune response comprises antibodies with a plurality of different heavy chain variable domains but only a single light chain variable domain.

The design and development of a new common light chain suitable for fitting to de-novo generated antibodies is demanding. Thus, this approach is not deemed the first choice for developing recombinant, multispecific antibodies, as it is very likely that further optimization is required and sequence modifications have to be made.

Common light chains and methods to generate such common light chains are reported, e.g., in WO 98/50431, WO 2010/084197, US 2013/045492, WO 2011/097603 and WO 2012/148873.

In WO 2004/009618 a common VL is reported in SEQ ID NO: 1 (comprised in UBS54 and K53). In SEQ ID NO: 18 a common light chain obtained from phages directed against CD22 (clone B28), CD72 (clone 11-2) and HLA-DR (class II; clone 1-2) is reported.

In US 2007/098712 common VL sequences of anti-Ob-R antibody clone 26 and anti-HER3 antibody clone 18 were used to construct a bispecific antibody. Also reported is that the anti-Mp1 scFv 12B5 (GenBank accession number AF048775) and the anti-HER3 scFv clone H6 (GenBank accession number AF048774) utilize identical VL sequences and substantially different VH sequences.

In WO 2010/84197 a recombinant antibody comprising a heavy chain and a light chain, wherein the light chain comprises the sequence as set forth in SEQ ID NO: 8 is reported. SEQ ID NO: 8 is the amino acid sequence of V-segment VKVI-2-1-(1)-A14 (IGKV6D-41*01). Further amino acid sequences of common light chains are reported in SEQ ID NO: 12 to 14.

Another common light chain approach is reported in US 2010/0331527, wherein two antibodies of different specificity use the same light chain.

In WO 2011/097603 engineered human Vkappa and Vlambda common light chains based on the human Vkappa 1-39Jkappa 5 locus, the human Vkappa 3-20Jkappa 1 locus and the human VpreBJlambda 5 locus are reported.

Common light chains and methods for making them are reported in US 2012/0192300, US 2012/021409, US 2011/0195454, and US 2013/0045492.

In WO 2012/018764 genetically modified mice and methods for making and using them are reported, wherein the mice comprise a replacement of all or substantially all immunoglobulin heavy chain V gene segments, D gene segments, and J gene segments with at least one light chain V gene segment and at least one light chain J gene segment.

In WO 2013/157953 a germline-like common light chain derived from the rearranged germline human kappa light chain IgVK1-39/JK or IGVK3-20/JK is reported.

In WO 2014/22540 it is outlined that a universal light chain can be a κ light chain selected from a VK1-39 and a VK3-20 light chain or a λ light chain selected from a VL1-40 and a VL2-14 light chain. In a specific embodiment the human VL gene segment is a human VK1-39JK5 gene segment or a human VK3-20JK1 gene segment.

In WO 2014/51433 the common light chain 012 is reported, which is the human rearranged kappa light chain IgVK1-39*01/IgJK1*01. This sequence is a germline sequence that is frequently used in the human repertoire and has superior ability to pair with many different VH regions, and has good thermodynamic stability, yield and solubility.

In US 2015/037337 it is reported that human JH6*02 is a common, conserved variant in humans, and thus a good candidate for construction of a transgenic IgH locus.

In WO 2015/052230 in SEQ ID NO: 6 the amino acid sequence of modified heavy chain CH3-CH2-CH1-VL, wherein VL is a variable domain of a common light chain (CLC-Fc cross-MAb), is reported.

In WO 2015/153765 common light chains are reported in the N-term-VL-CK-C-term fusion polypeptides of SEQ ID NO: 78 and 79.

Transgenic rabbits comprising a human immunoglobulin locus are reported in WO 2000/46251, WO 2002/12437, WO 2005/007696, WO 2006/047367, US 2007/0033661, and WO 2008/027986.

### SUMMARY OF THE INVENTION

One aspect as reported herein is a common antibody light chain variable domain that has the amino acid sequence

(SEQ ID NO: 01)
DIQMTQSPSS LSASVGDRVT ITCRASQSIS SYLNWYQQKP
GKAPKLLIYA ASSLQSGVPS RFSGSGSGTD FTLTISSLQP
EDFATYYCQQ SYSTPLTFGG GTKVEIK

or a variant thereof.

One aspect as reported herein is a common antibody light chain comprising a light chain variable domain that has the amino acid sequence

(SEQ ID NO: 01)
DIQMTQSPSS LSASVGDRVT ITCRASQSIS SYLNWYQQKP
GKAPKLLIYA ASSLQSGVPS RFSGSGSGTD FTLTISSLQP
EDFATYYCQQ SYSTPLTFGG GTKVEIK

or a variant thereof.

In one embodiment the common light chain comprises up to 13 amino acid mutations. In one preferred embodiment the common light chain comprises up to 13 amino acid mutations, whereof at most 11 are in the HVRs.

In one embodiment the common light chain comprises up to 11 amino acid mutations.

In one embodiment the common light chain comprises 1 to 11 amino acid mutations within the amino acid sequence of SEQ ID NO: 01. In one preferred embodiment the common light chain comprises 1 to 13 amino acid mutations within the amino acid sequence of SEQ ID NO: 01, whereof at most 11 mutations are in the HVRs.

In one embodiment a variant of the common antibody light chain as reported herein comprises a light chain variable domain that has a sequence identity to SEQ ID NO: 01 of 90% or more (i.e. comprises up to 11 mutations). In one embodiment the sequence identity is 95% or more. In one embodiment the sequence identity is 98% or more.

One aspect as reported herein is an antibody comprising a light chain as reported herein.

One aspect as reported herein is a multispecific antibody comprising two or more different heavy chain variable domains and two or more common light chain variable domains as reported herein.

In one embodiment the multispecific antibody is a bispecific full-length antibody comprising two different heavy chains and two common light chain variable domains or two common antibody light chains as reported herein.

In one embodiment the multispecific antibody is a trispecific antibody comprising three different heavy chain variable domains and three common light chain variable domains as reported herein.

In one embodiment the multispecific antibody is a tetraspecific antibody comprising four different heavy chain variable domains and four common light chain variable domains as reported herein.

One aspect as reported herein is the use of a common antibody light chain as reported herein for the generation of bispecific antibodies.

In one embodiment the use is by combining two common antibody light chains with a first antibody heavy chain and a second antibody heavy chain, wherein the first antibody heavy chain together with a common antibody light chain forms a first antigen binding site and the second antibody heavy chain together with a common antibody light chain forms a second antigen binding site.

One aspect as reported herein is a transgenic vector comprising a humanized immunoglobulin light chain locus, wherein said humanized immunoglobulin light chain locus comprises
(a) a V gene segment derived from human light chain V segment IGKV1-39-01,
(b) 3′ proximal to said light chain gene segment a promoter, and
(c) 5′ proximal to said light chain gene segment at least a fragment of the human IGKJ4 J-element.

In one embodiment transgenic vector comprises a humanized light chain locus, wherein said humanized light chain locus comprises
(a) as V gene segment the human light chain V segment IGKV1-39-01,
(b) 3′ proximal to said light chain gene segment a promoter, and
(c) 5′ proximal to said light chain gene segment the human IGKJ4 J-element or a functional fragment thereof.

One aspect as reported herein is a transgenic rabbit comprising the humanized immunoglobulin light chain locus present in the transgenic vector as reported herein. In one embodiment the transgenic rabbit has an essentially intact endogenous regulatory and antibody production machinery.

In one embodiment the transgenic rabbit further comprises
(1) a transgene derived from the rabbit immunoglobulin heavy chain locus, substituted with 8 human VH elements, human JH1-JH6 elements, human Cμ-coding regions fused to human bcl2 coding sequence, and human Cγ coding regions;
(2) a transgene derived from the rabbit immunoglobulin light chain locus, comprising the human Vκ element IGKV1-39-01 and the human IgKJ4 J-element;

(3) transgenes derived from the human CD79α and CD79β loci; and
(4) loss-of-function mutations within the rabbit Cμ and rabbit Cκ loci.

One aspect as reported herein is a B-cell from the transgenic rabbit as reported herein, comprising the humanized immunoglobulin light chain locus present in the transgenic vector as reported herein.

One aspect as reported herein is a method for producing a human immunoglobulin using the transgenic rabbit as reported herein.

In one embodiment the human immunoglobulin is an antibody. In one embodiment the human immunoglobulin is a polyclonal antibody. In one preferred embodiment the human immunoglobulin is a monoclonal antibody.

### DETAILED DESCRIPTION OF THE INVENTION

### Definitions

The term “common light chain variable domain” as used herein denotes a specific antibody light chain variable domain amino acid sequence that can pair with different antibody heavy chain variable domain amino acid sequence to form a functional antigen binding site of different specificities, i.e. bind to different epitopes either on the same antigen or on different antigens. The common light chain variable domain has in one embodiment an amino acid sequence identity of at least 80%, or at least 90%, or at least 95%, or in a preferred embodiment more than 98% to SEQ ID NO: 01. The amino acid residue differences normally have only little or even no effect on antigen binding. Thus, the term “common light chain variable domain” also encompasses antibody light chain variable domains which have some minor amino acid sequence differences but which when paired with the same heavy chain of an antibody form a binding site of the same specificity and comparable affinity.

It is possible to identify common light chain variable domains which on the one hand are not identical but on the other hand are functionally equivalent. This is possible, for example, by introducing and testing conservative amino acid mutations, changes of amino acids residues in parts of the common light chain that do not or only slightly influence the binding specificity of the binding site when the common light chain is paired with an antibody heavy chain variable domain.

“Operably linked” refers to a juxtaposition of two or more components, wherein the components so described are in a relationship permitting them to function in their intended manner. For example, a promoter and/or enhancer are operably linked to a coding sequence, if it acts in cis to control or modulate the transcription of the linked sequence. Generally, but not necessarily, the DNA sequences that are “operably linked” are contiguous and, where necessary to join two protein encoding regions such as a secretory leader and a polypeptide, contiguous and in (reading) frame. However, although an operably linked promoter is generally located upstream of the coding sequence, it is not necessarily contiguous with it. Enhancers do not have to be contiguous. An enhancer is operably linked to a coding sequence if the enhancer increases transcription of the coding sequence. Operably linked enhancers can be located upstream, within or downstream of coding sequences and at considerable distance from the promoter. A polyadenylation site is operably linked to a coding sequence if it is located at the downstream end of the coding sequence such that transcription proceeds through the coding sequence into the polyadenylation sequence. A translation stop codon is operably linked to an exonic nucleic acid sequence if it is located at the downstream end (3′ end) of the coding sequence such that translation proceeds through the coding sequence to the stop codon and is terminated there. Linking is accomplished by recombinant methods known in the art, e.g., using PCR methodology and/or by ligation at convenient restriction sites. If convenient restriction sites do not exist, then synthetic oligonucleotide adaptors or linkers are used in accord with conventional practice.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman, S. et al., J. Chromatogr. B 848 (2007) 79-87.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

Antibody Generation in Mammals

Antibody gene generation (see Molecular Biology of the Cell. 4th edition. Alberts B, Johnson A, Lewis J, et al. New York: Garland Science; 2002; and Immunobiology: The Immune System in Health and Disease. 5th edition. Janeway, C. A. Jr, Travers P, Walport M, et al. New York: Garland Science; 2001):

The genetic locus for the λ light chain (chromosome 22) has about 30 functional Vλ gene segments and four pairs of functional Jλ gene segments and Cλ genes. The κ locus (chromosome 2) is organized in a similar way, with about 40 functional Vκ gene segments accompanied by a cluster of five Jκ gene segments but with a single Cκ gene. In approximately 50% of individuals, the entire cluster of κV gene segments has undergone an increase by duplication. The heavy-chain locus (chromosome 14) has about 65 functional VH gene segments and a cluster of around 27 D segments lying between these VH gene segments and six JH gene segments. The heavy-chain locus also contains a large cluster of CH genes. The total length of the heavy-chain locus is over 2 megabases (2 million bases), whereas some of the D segments are only six bases long.

The V region, or V domain, of an immunoglobulin heavy or light chain is encoded by more than one gene segment. For the light chain, the V domain is encoded by two separate DNA segments. The first segment encodes the first 95-101 amino acids of the light chain and is termed a V gene segment because it encodes most of the V domain. The second segment encodes the remainder of the V domain (up to 13 amino acids) and is termed a joining or J gene segment. Thus, of the three hypervariable loops in the variable domains of immunoglobulins, two are encoded within the V gene segment DNA, whereas the third (HV3 or CDR3) falls at the joint between the V gene segment and the J gene segment, and in the heavy chain is partially encoded by the D gene segment. In both heavy and light chains, the diversity of CDR3 is significantly increased by the addition and deletion of nucleotides at two steps in the formation of the junctions between gene segments. The added nucleotides are known as P-nucleotides and N-nucleotides.

During B-cell development, the V and J gene segments (for the light chain) and the V, D, and J gene segments (for the heavy chain) are joined together to form a functional VL- or VH-region coding sequence by a process of site-specific recombination called V(D)J joining. Conserved DNA sequences flank each gene segment and serve as recognition sites for the joining process, ensuring that only appropriate gene segments recombine. Thus, for example, a V segment will always join to a J or D segment but not to another V segment. Joining is mediated by an enzyme complex called the V(D)J recombinase. This complex contains two proteins that are specific to developing lymphocytes, as well as enzymes that help repair damaged DNA in all our cells.

Any of the 40 V segments in the human κ light-chain gene-segment pool, for example, can be joined to any of the 5 J segments, so that at least 200 (40×5) different κ-chain V regions can be encoded by this pool. Similarly, any of the 51 V segments in the human heavy-chain pool can be joined to any of the 6 J segments and any of the 27 D segments to encode at least 8262 (51×6×27) different heavy-chain V regions.

The combinatorial diversification resulting from the assembly of different combinations of inherited V, J, and D gene segments just discussed is an important mechanism for diversifying the antigen-binding sites of antibodies. By this mechanism alone, a human can produce 287 different VL regions (200κ and 116λ) and 8262 different VH regions.

In most cases of site-specific recombination, DNA joining is precise. But during the joining of antibody (and T cell receptor) gene segments, a variable number of nucleotides are often lost from the ends of the recombining gene segments, and one or more randomly chosen nucleotides may also be inserted. This random loss and gain of nucleotides at joining sites is called junctional diversification, and it enormously increases the diversity of V-region coding sequences created by recombination, specifically in the third hypervariable region.

The Common Light Chain as Reported Herein

Herein is reported a humanized light chain locus.

The invention is based at least in part on the finding that a humanized light chain immunoglobulin locus comprising multiple V gene elements but only single V gene element combined with a promoter can be used as common light chain locus in a transgenic rabbit.

The humanized light chain locus as reported herein comprises
(a) a V gene segment derived from human light chain V segment IGKV1-39-01,
(b) 3′ proximal to said light chain gene segment a promoter, and
(c) 5′ proximal to said light chain gene segment at least a fragment of the human IGKJ4 J-element.

The complete light chain V gene segment IGKV1-39-01 has the following nucleic acid sequence (see e.g. GenBank X93627, Homo sapiens germline immunoglobulin kappa light chain, variable region (DPK9); 287 bp; SEQ ID NO: 02):

gacatccaga tgacccagtc tccatcctcc ctgtctgcat
ctgtaggaga cagagtcacc atcacttgcc gggcaagtca
gagcattagc agctatttaa attggtatca gcagaaacca
gggaaagccc ctaagctcct gatctatgct gcatccagtt
tgcaaagtgg ggtcccatca aggttcagtg gcagtggatc
tgggacagat ttcactctca ccatcagcag tctgcaacct
gaagattttg caacttacta ctgtcaacag agttacagta
cccctcc.

The corresponding amino acid sequence is (SEQ ID NO: 03):

DIQMTQSPSS LSASVGDRVT ITCRASQSIS SYLNWYQQKP
GKAPKLLIYA ASSLQSGVPS RFSGSGSGTD FTLTISSLQP
EDFATYYCQQ SYSTP

The full-length human IgKJ4*01/02 has the following nucleic acid (SEQ ID NO: 04) and amino acid (SEQ ID NO: 05) sequences:

| | |
|---|---|
| nucleic acid: | ctcactttcggcggagggaccaaggtggagatcaaa |
| amino acid: | L T F G G G T K V E I K |

The use of a common light chain enables the generation of multispecific antibodies (e.g. bispecific full length antibodies) by combining different heavy chain variable domains, each binding to a different epitope/antigen/target with the same light chain variable domain or the same variant thereof and thereby reducing the side-product complexity.

In one embodiment the humanized light chain locus comprises 25 to 30 human Vκ elements and a human Cκ coding region, wherein
(a) the 3′ proximal Vκ element is a V gene segment derived from human light chain V segment IGKV1-39-01,
(b) to said 3′ proximal light chain gene segment (3′ proximal) a promoter is operably linked, and
(c) 5′ proximal to said light chain gene segment at least a fragment of the human IGKJ4 J-element is operably linked.

In one embodiment the promoter is a human kappa variable region promoter (subgroup V kappa I).

In one embodiment the V gene segment comprises a human kappa immunoglobulin light chain leader peptide encoding nucleic acid. In one embodiment the leader peptide has the amino acid sequence of SEQ ID NO: 15.

In one embodiment the V gene segment comprises a human kappa immunoglobulin leader peptide encoding nucleic acid and a chicken derived spacer sequence between the leader peptide encoding nucleic acid sequence and the V gene segment. In one embodiment the chicken derived spacer sequence is SEQ ID NO: 16.

The light chain immunoglobulin locus encodes the following light chain V-segment (SEQ ID NO: 03, HVRs underlined):

DIQMTQSPSS LSASVGDRVT ITCRASQSIS SYLNWYQQKP
GKAPKLLIYA ASSLQSGVPS RFSGSGSGTD FTLTISSLQP
EDFATYYCQQ SYSTP

and the following human J-element (SEQ ID NO: 05, part of the HVR-L3 is underlined):

LTFGG GTKVEIK.

Thus, one aspect as reported herein is an antibody light chain that comprises a light chain variable domain with the amino acid sequence

DIQMTQSPSS LSASVGDRVT ITCRASQSIS SYLNWYQQKP
GKAPKLLIYA ASSLQSGVPS RFSGSGSGTD FTLTISSLQP
EDFATYYCQQ SYSTPLTFGG GTKVEIK

or a variant thereof.

Also encompassed herein are variants of this amino acid sequence that arise due to gene conversion and hypermutation in the rabbit during B-cell maturation.

In one embodiment the (mature) light chain comprises 1 to 4 amino acid mutations with respect to the light chain encoded by the light chain immunoglobulin locus outside the HVRs.

In one embodiment the (mature) light chain comprises 1 to 15 amino acid mutations with respect to the light chain encoded by the light chain immunoglobulin locus.

In one embodiment the (mature) light chain comprises 1 to 11 amino acid mutations with respect to the light chain encoded by the light chain immunoglobulin locus.

In one embodiment the (mature) light chain comprises 1 to 15 amino acid mutations with respect to the light chain encoded by the light chain immunoglobulin locus, whereof at most 11 are in the HVRs.

One aspect as reported herein is a bispecific full-length antibody comprising two different heavy chains and two light chains, whereby the light chains are identical and the variable domains have an amino acid sequence as reported herein.

Transgenic Rabbits

The light chain locus as reported herein can be used in the generation of human immunoglobulin producing transgenic rabbits.

Thus, one aspect as reported herein is a light chain transgenic rabbit with a humanized immunoglobulin light chain locus as reported herein.

The transgenic rabbit has a humanized immunoglobulin locus and still has the antibody maturation process of a wild-type rabbit, using e.g. gene conversion in order to generate antibody diversity. Therefore the heavy chain and light chain loci of a wild-type rabbit have been inactivated and respective humanized immunoglobulin transgene loci have been introduced into the genome of the rabbit enabling the rabbit to produce human(ized)/human-like antibodies. The genotype of the transgenic rabbit can be described as follows:
the transgenic rabbit comprises
   (1) a transgene derived from the rabbit immunoglobulin heavy chain locus, substituted with 8 human VH elements, human JH1-JH6 elements, human Cμ-coding regions fused to human bcl2 coding sequence, and human Cy coding regions;
   (2) a transgene derived from the rabbit immunoglobulin light chain locus, comprising the human Vκ element IGKV1-39-01 and the human IgKJ4 J-element;
   (3) transgenes derived from the human CD79α and CD79β loci; and
   (4) loss-of-function mutations within the rabbit Cμ and rabbit Cκ loci.

Herein is reported a transgenic rabbit comprising a humanized immunoglobulin heavy chain locus and a humanized immunoglobulin light chain locus, wherein
i) the humanized heavy chain immunoglobulin locus is derived from an immunoglobulin locus or a portion of an immunoglobulin locus of a rabbit, and comprises multiple immunoglobulin heavy chain gene segments wherein
   (a) at least one of said heavy chain gene segments is a human heavy chain V segment of the VH3 family as 3′ proximal V gene segment flanked by nucleotide sequences comprising (between 20 and 1000 contiguous nucleotides from) a rabbit spacer sequence of SEQ ID NO: 06,
   (b) said gene segments are juxtaposed in an unrearranged, or partially rearranged, or fully rearranged configuration, and
   (c) said humanized immunoglobulin locus is capable of undergoing gene rearrangement, if necessary, and gene conversion and/or hypermutation, and producing a repertoire of humanized immunoglobulins in said rabbit, and
ii) the humanized light chain immunoglobulin locus comprises
   (a) a V gene segment derived from human light chain V segment IGKV1-39-01,
   (b) 3′ proximal to said light chain gene segment a promoter, and
   (c) 5′ proximal to said light chain gene segment at least a fragment of the human IGKJ4 J-element.

In one embodiment the transgenic rabbit is homozygous for the humanized heavy chain locus and the humanized light chain locus.

In one embodiment the transgenic rabbit is heterozygous for the humanized heavy chain locus and the humanized light chain locus.

In one embodiment the transgenic rabbit is inactivated for endogenous antibody heavy chain expression and/or endogenous antibody light chain expression.

One aspect as reported herein is a B-cell from the transgenic rabbit as reported herein comprising the humanized light chain immunoglobulin locus as reported herein.

One aspect as reported herein is an isolated B-cell comprising the humanized light chain immunoglobulin locus as reported herein.

In one embodiment the B-cell further comprises a humanized heavy chain immunoglobulin locus that is derived from an immunoglobulin locus or a portion of an immunoglobulin locus of a rabbit, comprising multiple immunoglobulin heavy chain gene segments wherein
(a) at least one of said heavy chain gene segments is a human heavy chain V segment of the VH3 family flanked by nucleotide sequences comprising (between 20 and 1000 contiguous nucleotides from) a rabbit spacer sequence of SEQ ID NO: 06,
(b) said gene segments are juxtaposed in an unrearranged, partially rearranged or fully rearranged configuration, and
(c) said humanized immunoglobulin locus is capable of undergoing gene rearrangement, if necessary, and gene conversion and/or hypermutation, and producing a repertoire of human immunoglobulins in said rabbit.

Also an aspect as reported herein is a method for producing a human immunoglobulin using the transgenic rabbit as reported herein.

In one embodiment the human immunoglobulin is obtained from the blood of the rabbit.

Herein is reported a rabbit having a genome comprising a modification of the heavy chain immunoglobulin locus and the light chain immunoglobulin locus, wherein the modification is the inactivation of the endogenous rabbit immunoglobulin loci and the introduction of humanized immunoglobulin loci, resulting in a transgenic rabbit. The genome of the transgenic rabbit, thus, comprises exogenous nucleic acid sequences encoding different human immunoglobulin heavy chain variable domains and a (single functional) human immunoglobulin light chain variable domain.

The humanized immunoglobulin loci, i.e. the respective nucleic acid sequences, are integrated into the rabbit genome. The modification of the immunoglobulin loci is an insertion of one or more transgenic human immunoglobulin gene segments sequences with the concomitant inactivation of the respective one or more endogenous rabbit immunoglobulin gene segments.

The term “humanized immunoglobulin locus” denotes an isolated immunoglobulin locus comprising one or more human elements, such as one or more V-regions and/or none, and/or one or more J-elements. These are combined with exogeneous elements, i.e. combined with genetic elements not combined therewith in nature, such as promoters and/or regulatory elements from non-human organisms.

The transgenic rabbit as reported herein can be used for the generation of human antibodies. Thus, one aspect as reported herein is an (isolated) B-cell or (isolated) tissue from a transgenic rabbit as reported herein.

Also an aspect as reported herein is the use of a transgenic rabbit as reported herein for the generation of either (i) a chimeric antibody comprising human heavy chain and light chain variable regions and rabbit constant regions, or (ii) a fully human antibody.

An aspect as reported herein is a method for producing an antibody specifically binding to an antigen comprising the steps of:
(a) immunizing a transgenic rabbit as reported herein (with the antigen),
(b) isolating at least one cell from the immunized transgenic rabbit producing an antibody specifically binding the antigen,
(c) culturing the at least one cell of step (b) as single deposited cell to produce the antibody.

In one embodiment the at least one cell obtained in step b) is a splenocyte. In one embodiment the at least one cell obtained in step b) is a B-cell.

Also an aspect as reported herein is a method for producing an antibody specifically binding to an antigen (of interest) comprising the steps of:
(a) providing one or more B-cell(s) from a transgenic rabbit as reported herein, which had been immunized with the antigen (of interest),
(b) culturing the at least one or more B-cell(s) of step (a) as single deposited cell to produce the antibody.

Also an aspect as reported herein is a method for producing an antibody specifically binding to an antigen comprising the steps of:
(a) cultivating a mammalian cell comprising a nucleic acid encoding an antibody specifically binding to the antigen, wherein at least the nucleic acid encoding the variable domains as the antibody had been obtained from a transgenic rabbit as reported herein that had been immunized with the antigen,
(b) recovering the antibody from the mammalian cell or the cultivation medium.

In one embodiment the antibody is a monoclonal antibody.

In one embodiment the immunizing is with the antigen, with DNA encoding the antigen, with the antigen and DNA encoding the antigen, or with cells expressing the antigen.

In one embodiment the immunizing is performed by administering the antigen, DNA encoding the antigen, the antigen together with DNA encoding the antigen, or cells expressing the antigen to the transgenic rabbit as reported herein.

The following examples and sequences are provided to aid the understanding of the present invention, the true scope of which is set forth in the appended claims. It is understood that modifications can be made in the procedures set forth without departing from the spirit of the invention.

### Example 1

Immunization of Rabbits

The transgenic rabbits used for immunization contained (1) a transgene derived from the rabbit immunoglobulin heavy chain locus, substituted with 8 human VH elements, human JH1-JH6 elements, human Cμ-coding regions fused to human bcl2 coding sequence, and human Cγ coding regions; (2) a transgene derived from the rabbit immunoglobulin light chain locus, substituted with 25 human Vκ elements, the proximal Vκ element fused to human Jκ4, and a human Cκ coding region; (3) transgenes derived from the human CD79a and CD79b loci; and (4) loss-of-function mutations within the rabbit Cμ and rabbit Cκ loci.

Protein Immunization

Rabbits were immunized with 400 μg recombinant soluble antigen, emulsified with complete Freund's adjuvant, at day 0 by intradermal application, and with 200 μg each of antigen, emulsified with complete Freund's adjuvant, at days 7, 14, 42, 70 and 84 or 98, by alternating intramuscular and subcutaneous applications. Blood (10% of estimated total blood volume) was taken at around days 20-21, 34-48, 62-76 and 90-104. Serum was prepared, which was used for titer determination by ELISA, and peripheral mononuclear cells were isolated, which were used as a source of antigen-specific B-cells in the B-cell cloning process. Accordingly human antibodies were obtained.

DNA Immunization

Rabbits were immunized genetically, using a plasmid expression vector coding for full-length antigen, by intradermal application of 400 μg vector DNA, followed by electroporation (5 square pulses of 750 V/cm, duration 10 ms, interval 1 s). Rabbits received 7 consecutive immunizations at days 0, 14, 28, 49, 70, 98 and 126. Blood (10% of estimated total blood volume) was taken at days 35, 77, 105 and 133. Serum was prepared, which was used for titer determination by ELISA, and peripheral mononuclear cells were isolated, which were used as a source of antigen-specific B-cells in the B-cell cloning process.

### Example 2

Determination of Serum Titers

Antigen was immobilized on a 96-well NUNC Maxisorb plate at 1.75-2 μg/ml, 100 μl/well, in PBS, followed by: blocking of the plate with 2% CroteinC in PBS, 200 μl/well; application of serial dilutions of antisera, in duplicates, in 0.5% CroteinC in PBS, 100 μl/well; detection with either (1) HRP-conjugated donkey anti-rabbit IgG antibody (Jackson Immunoresearch), or (2) HRP-conjugated rabbit anti-human IgG antibody (Pierce/Thermo Scientific; 1/5000), or (3) biotinylated goat anti-human kappa antibody (Southern Biotech/Biozol; 1/5000) and streptavidin-HRP; each diluted in 0.5% CroteinC in PBS, 100 μl/well. For all steps, plates were incubated for 1 h at 37° C. Between all steps, plates were washed 3-times with 0.05% Tween 20 in PBS. Signal was developed by addition of BM Blue POD Substrate soluble (Roche), 100 μl/well; and stopped by addition of 1 M HCl, 100 μl/well. Absorbance was read out at 450 nm, against 690 nm as reference. Titer was defined as dilution of antisera resulting in half-maximal signal.

### Example 3

B-Cell Cloning and Sorting

Isolation of Rabbit Peripheral Blood Mononuclear Cells (PBMC)

Transgenic rabbits of Example 1 were used as a source of blood. EDTA containing whole blood was diluted two-fold with 1×PBS before density centrifugation on lympholyte mammal (Cedarlane Laboratories, Burlington, Ontario, Canada) according to the specifications of the manufacturer. PBMCs were washed twice with 1×PBS before staining with antibodies.

EL-4 B5 Medium

RPMI 1640 (Pan Biotech, Aidenbach, Germany) supplemented with 10% FCS (Hyclone, Logan, Utah, USA), 2 mM Glutamine, 1% penicillin/streptomycin solution (PAA, Pasching, Austria), 2 mM sodium pyruvate, 10 mM HEPES (PAN Biotech, Aidenbach, Germany) and 0.05 mM (3-mercaptoethanol (Gibco, Paisley, Scotland).

Depletion of Macrophages/Monocytes

Sterile 6-well plates (cell culture grade) were used to deplete macrophages and monocytes through unspecific adhesion. Each well was filled at maximum with 4 ml media and up to 6×10⁶ peripheral blood mononuclear cells from the immunized rabbit and allowed to bind for 1 h at 37° C. and 5% CO₂ in the incubator. The cells in the supernatant were used for the antigen panning step.

Coating of Plates

Sterile cell culture 6-well plates were coated with 2 μg/ml antigen protein, or sterile streptavidin coated 6-well plates (Microcoat, Bernried, Germany) were coated with 2 μg/ml biotinylated antigen for 3 hours at room temperature or overnight at 4° C. Plates were washed in sterile PBS three times before use.

Enrichment of B Cells on the Antigen Protein

6-well tissue culture plates coated with antigen protein were seeded with up to 6×10⁶ cells per 4 ml medium and allowed to bind for 1 h at 37° C. and 5% CO₂ in the incubator. After the enrichment step on antigen protein non-adherent cells were removed by carefully washing the wells 1-2 times with 1×PBS. The remaining sticky cells were detached by trypsin for 10 min. at 37° C. in the incubator. Trypsination was stopped with EL-4 B5 medium. Then the cells were washed twice in media. The cells were kept on ice until the immune fluorescence staining.

Immune Fluorescence Staining and Flow Cytometry

Anti-IgG FITC antibody (AbD Serotec, Dusseldorf, Germany) was used for single cell sorting. For surface staining, cells from the depletion and enrichment step were incubated with the anti-IgG FITC antibody in PBS for 30-45 min. rolling in the cold room at 4° C. in the dark. Following centrifugation, the supernatants were removed by aspiration. The PBMCs were subjected to 2 cycles of centrifugation and washing with ice cold PBS. Finally the PBMCs were resuspended in ice cold PBS and immediately subjected to the FACS analyses. Propidium iodide in a concentration of 5 μg/ml (BD Pharmingen, San Diego, Calif., USA) was added prior to the FACS analyses to discriminate between dead and live cells.

A Becton Dickinson FACSAria equipped with a computer and the FACSDiva software (BD Biosciences, USA) were used for single cell sort.

B-Cell Cultivation

The cultivation of the rabbit B-cells was done by a method described by Seeber, S., et al., PLoS One 9 (2014) e86184. Briefly, single sorted rabbit B-cells were incubated in 96-well plates with 200 μl/well EL-4 B5 medium containing Pansorbin cells (1:100,000) (Calbiochem (Merck), Darmstadt, Deutschland), 5% rabbit thymocyte supernatant (MicroCoat, Bernried, Germany) and gamma-irradiated murine EL-4 B5 thymoma cells (2.5×10e⁴ cells/well) for 7 days at 37° C. in the incubator. The supernatants of the B-cell cultivation were removed for screening and the remaining cells were harvested immediately and were frozen at −80° C. in 100 μl RLT buffer (Qiagen, Hilden, Germany).

### Example 4

B-Cell PCR

Total RNA was prepared from B-cells lysate (resuspended in RLT buffer) using the NucleoSpin 8/96 RNA kit (Macherey&Nagel) according to manufacturer's protocol. RNA was eluted with 60 μl RNase free water. 6 μl of RNA was used to generate cDNA by reverse transcriptase reaction using the Superscript III First-Strand Synthesis SuperMix (Invitrogen) and an oligo dT-primer according to the manufacturer's instructions. All steps were performed on a Hamilton ML Star System. 4 μl of cDNA were used to amplify the immunoglobulin heavy and light chain variable regions (VH and VL) with the AccuPrime SuperMix (Invitrogen) in a final volume of 50 μl using the primers rbHC.up and rbHC.do for the heavy chain and BcPCR_FHLC leader.fw and BcPCR_huCkappa.rev for the light chain. All forward primers were specific for the signal peptide (of respectively VH and VL) whereas the reverse primers were specific for the constant regions (of respectively VH and VL). The PCR conditions for the RbVH+RbVL were as follows: Hot start at 94° C. for 5 min.; 35 cycles of 20 sec. at 94° C., 20 sec. at 70° C., 45 sec. at 68° C., and a final extension at 68° C. for 7 min. The PCR conditions for the HuVL were as follows: Hot start at 94° C. for 5 min.; 40 cycles of 20 sec. at 94° C., 20 sec. at 52° C., 45 sec. at 68° C., and a final extension at 68° C. for 7 min.

Primer Sequences:

rbHC.up
(SEQ ID NO: 07)
AAGCTTGCCACCATGGAGACTGGGCTGCGCTGGCTTC
rbHCf.do
(SEQ ID NO: 08)
CCATTGGTGAGGGTGCCCGAG
BcPCR_FHLC_leader.fw
(SEQ ID NO: 09)
ATGGACATGAGGGTCCCCGC
BcPCR_huCkappa.rev
(SEQ ID NO: 10)
GATTTCAACTGCTCATCAGATGGC

8 μl of 50 μl PCR solution were loaded on a 48 E-Gel 2% (Invitrogen G8008-02). Positive PCR reactions were cleaned using the NucleoSpin Extract II kit (Macherey&Nagel; 740609250) according to manufacturer's protocol and eluted in 50 μl elution buffer. All cleaning steps were performed on a Hamilton ML Starlet System.

The used antigen was the extracellular domain of TPBG (trophoblast glycoprotein, SEQ ID NO: 11).

The resulting antibodies for the extracellular domain of TPBG have the following light chain variable domains:

051 (SEQ ID NO: 12):
DIQMTQSPSS VSASVGDRVT ITCRASQGIY SWLAWYQQKP
GKAPKLLIYA ASSLQSGVPS RFSGSGSGTD FTLTISSLQP
EDFATYYCQQ SDSPPYTFGQ GTKLEIK,
091 (SEQ ID NO: 13):
DIQMTQSPSS LSASVGDRVT ITCQASQDIS NYLNWYQQKP
GKAPKLLIYA ASTLQIGVPS RFSGSGSGTD FTFTISSLQP
EDFATYYCQQ ANSFPLTFGG GTKVEIK,
097 (SEQ ID NO: 14):
DIQMTQSPSS LSASVGDRVT ITCRASQSIS SYLNWYQQKP
GKAPKLLIYA ASSLQSGVPS RFSGSGSGTD FTLTISSLQP
EDFATYYCQQ SDSFPLTFGG GTKVEIK.

### Example 5

Binding of TPBG-Specific Fab Fragments to TPBG

To assess binding of recombinant TPBG, Nunc Maxisorb streptavidin coated plates (MicroCoat #11974998001) were coated with 25 μl/well biotinylated human TPBG-AviHis at a concentration of 100 ng/ml. Plates were incubated at 4° C. overnight. After washing (3×90 μl/well with PBST-buffer) anti-TPBG samples were added in a 1:2 dilution series starting at 2 μg/ml and incubated 1 h at RT. After washing (3×90 μl/well with PBST-buffer) 25 μl/well goat anti c-myc HRP (Bethyl, # A190-104P) or goat anti hu kappa HRP (Millipore, # AP502P) was added in a 1:7000 or 1:4000 dilution, respectively and incubated at RT for 1 h on a shaker. After washing (3×90 μl/well with PBST-buffer) 25 μl/well TMB substrate (Calbiochem, #CL07) was added and incubated 2 min. Measurement took place at 370/492 nm on a Safire2 reader (Tecan).

To assess cellular binding of human TPBG, the human breast cancer tumor cell line MFC7 endogenously expressing TPBG was seeded at a concentration of 21000 cells/well in 384-well cellcoat Poly-D-Lysine plates (Greiner, #781940). Cells were allowed to attach over night at 37° C. After removing the supernatant, 25 μl/well of supernatant containing anti-TPBG antibodies were added in a 1:2 dilution series starting at 5 μg/ml and incubated 1 h at 4° C. Upon washing (2×50 μl/well PBST) cells were fixed by adding 50 μl/well 0.05% Glutaraldehyde (Sigma, 25%) diluted in 1×PBS-buffer and incubated for 10 min at RT. After washing (3 times; 90 μl/well PBS-T), 25 μl/well secondary antibody was added for detection: goat anti c-myc HRP (1:5000, Bethyl) followed by 1 h incubation at room temperature on a shaker. After washing (3 times; 90 μl/well PBS-T) 25 μl/well TMB substrate solution (Calbiochem) was added. After 10 min at room temperature, measurement took place at 370/492 nm on a Safire2 reader (Tecan).

TABLE
Binding of anti-TPBG Fab fragments to human TPBG

| EC50 [ng/ml] | recombinant TPBG | MCF7 |
|---|---|---|
| 051 | 18.1 | 57.5 |
| 091 | 27.7 | 14.0 |
| 097 | 15.2 | 451.3 |

Fab fragments of 051, 091, and 097 were found to bind to human TPBG or recombinant source or expressed on cells of a human breast cancer cell line.

1. A method of generating a bispecific antibody comprising: use of a common antibody light chain comprising a variable domain that has the amino acid sequence of SEQ ID NO: 01 or is a variant thereof.

2. The method according to claim 1 wherein the method comprises combining two common antibody light chains with a first antibody heavy chain and a second antibody heavy chain, wherein the first antibody heavy chain together with a common antibody light chain forms a first antigen binding site and the second antibody heavy chain together with a common antibody light chain forms a second antigen binding site.

3. The method according to claim 1, wherein the common light chain comprises 1 to 11 amino acid mutations within the amino acid sequence of SEQ ID NO: 01.

4. The method according to claim 1, wherein the common light chain comprises 1 to 13 amino acid mutations within the amino acid sequence of SEQ ID NO: 01, whereof at most 11 mutations are in the HVRs.

5. A bispecific full-length antibody comprising two heavy chains and two common light chains each comprising a variable domain that has the amino acid sequence of SEQ ID NO: 01 or is a variant thereof.

6. A transgenic vector comprising a humanized light chain locus, wherein said humanized light chain locus comprises
(a) as V gene segment the human light chain V segment IGKV1-39-01,
(b) 3′ proximal to said light chain gene segment a promoter, and
(c) 5′ proximal to said light chain gene segment the human IGKJ4 J-element or a functional fragment thereof.

7. A transgenic rabbit, comprising the humanized immunoglobulin locus present in the transgenic vector according to claim 6.

8. The transgenic rabbit according to claim 7, wherein the transgenic rabbit further comprises
(1) a transgene derived from the rabbit immunoglobulin heavy chain locus, substituted with 8 human VH elements, human JH1-JH6 elements, human Cμ-coding regions fused to human bcl2 coding sequence, and human Cγ coding regions;
(2) a transgene derived from the rabbit immunoglobulin light chain locus, comprising the human Vκ element IGKV1-39-01 and the human IgKJ4 J-element;
(3) transgenes derived from the human CD79α and CD79β loci; and
(4) loss-of-function mutations within the rabbit Cμ and rabbit Cκ loci.

9. A B-cell from the transgenic rabbit according to claim 7.

10. A method for producing a human immunoglobulin using the transgenic rabbit of claim 7.

11. The method according to claim 10, wherein the human immunoglobulin is an antibody.

12. The method according to claim 10, wherein the human immunoglobulin is a polyclonal antibody.

13. The method according to claim 10, wherein the human immunoglobulin is a monoclonal antibody.
